(19) United States
(12) Patent Application Publication
(10) Pub. No.: US 2016/0058924 A1
(43) Pub. Date: Mar. 3, 2016

(54) CANNULA FOR RF LIPOSUCTION

(71) Applicant: MEDEX TECHNOLOGIES CO., LTD., Seoul (KR)

(72) Inventor: Min Sik KIM, Seoul (KR)

(21) Appl. No.: 14/476,540

(22) Filed: Sep. 3, 2014

(51) Int. Cl. A61M 1/00; A61B 18/14

(52) U.S. Cl. CPC A61M 1/008; A61B 18/1487; A61M 2202/08; A61B 2018/00464

(57) ABSTRACT

Provided is a cannula to more rapidly and efficiently accelerate the suction of the fat from a human body. The cannula has an empty space therein and a pipe shape to suck a fat in a skin. The cannula includes a handpiece coupled with a rear end coupled with the cannula, and a control box including a micro-controller and a power supply. First and second RF electrode plates are provided at an outer portion of an inner insulator in opposition to each other. A temperature sensor is interposed between the first and second RF electrode plates, and an outer insulator is formed on an outer peripheral portion of the first and second RF electrode plates and the temperature sensor. The first and second electrodes are alternately wound in a spiral shape around an inner insulator of the cannula and spaced apart from each other by a predetermined interval.

### STATEMENT REGARDING PRIOR DISCLOSURES

The present application is based on Korean Application No. 10-2012-0078112 which was filed on Jul. 18, 2012, and published on Jan. 28, 2014, as Korean Application Publication No. 10-2014-0011176, which does not qualify as prior art under AIA 35 U.S.C. 102(b)(1)(A). A copy of the Korean application publication is attached hereto.

### BACKGROUND OF THE INVENTION

1. Field of the Invention

The present invention relates to a cannula for RF liposuction, capable of easily sucking fat by generating deep heat inside a human body using RF power to melt or soften a fat layer at a suction target area.

2. Description of the Related Art

In general, liposuction refers to a procedure of forcibly discharging subcutaneous fat out of a human body by sucking the subcutaneous fat through vacuum without damage to nerves or lymphatic vessels.

As the level of living is improved, the intake of meat is increased. As transportations are developed, human beings less take exercise such as walking, and a greater amount of animal fat, which is less dissolved in a human body, is accumulated in the human body, which causes obesity.

Recently, the obesity is recognized as one of diseases, which especially cause various adult diseases, such as artery hardening or diabetes. If the fat accumulated in the human body is not dissolved through a regular exercise, the fat may be accumulated in a subcutaneous fatty organism or on the surface of an organ, so that the shape of a body is changed to cause severe unbalance in abdomens, thighs, calves, or forearms.

Meanwhile, recently, liposuction has been widely performed in which the fat accumulated in the human body is artificially removed to remove extra flab, so that a slim figure can be obtained. A liposuction device, which is medical equipment to suck the fat from the human body, includes a cannula to suck the fat under the skin of the human body, a handpiece connected with a rear end of the cannula, and a body to adjust the speed of the handpiece.

The cannula is molded in the shape of a pipe having an empty space therein, and the handpiece integrated with the cannula generates vacuum suction force, sucks fat, and discharges the fat out through a connection horse.

In particular, according to the related art, an electrode, which applies high frequency (RF, radio frequency) current to the cannula, directly applies the RF current supplied from an external control box to the cannula, so that the RF current can be adjusted, or a plurality of electrodes may be arranged in a pad to receive the RF current, so that the fat in the human body is discharged out by an additional liposuction device.

The cannula according to the related art propagates an RF signal into a human body so that the vibration of ion molecules or atoms is caused. Then, the molecules or the atoms collide with each other to generate heat energy, and the heat energy is accumulated according to radiation time to increase a temperature. Accordingly, if the temperature is adjusted by adjusting the power and the frequency of the RF signal, heating, dehydration, and destruction effects may be obtained in a body tissue, and the above effects may be used in a blood circulation principle or a fat removal principle.

Regarding the electrode according to the related art, a cannula having an empty space therein and a pipe shape serves as the electrode. Accordingly, if RF current is applied to the cannula, significantly high energy is supplied to generate high heat, so that the inner part of the human body may be thermally damaged. In order to avoid the thermal damage, as described in Korean Unexamined Patent Publication No. 10-2010-0135863, a coolant tube is installed in the cannula and a saline solution is supplied to the coolant tube to lower the high heat. Accordingly, the electrode type has a complex structure, and an additional coolant tube must be installed. Accordingly, when liposuction is performed, a user feels uncomfortable. In addition, since an additional liposuction tube must be installed, the above structure is significantly inappropriate for a cannula for liposuction.

In addition, RF current is supplied to a plurality of electrodes arranged in a pad, the pad is attached to an abdomen of a patient, and an additional cannula is inserted into a body to discharge fat, which is dissolved by deep heat in the body, out. However, the structure is complex in that the pad and the cannula must be separately provided. Even if RF current is applied through bipolar electrodes provided outside the abdomen of the patient, the deep heat may be significantly weak, and the liposuction effect may be slightly made.

As the related art, there is Korean Unexamined Patent Publication No. 10-2010-0135863.

### SUMMARY OF THE INVENTION

Accordingly, the present invention has been made keeping in mind the above problems occurring in the related art, and an object of the present invention is to more rapidly and efficiently accelerate the removal of a fatty tissue by installing electrodes, which are operated at a bipolar mode, into a cannula to be inserted into a body of a patient.

To accomplish these objects, according to one aspect of the present invention, there is provided a cannula having an empty space therein and a pipe shape to suck a fat in a skin of a human body. The cannula includes a handpiece coupled with a rear end coupled with the cannula, and a control box equipped with a micro-controller and a power supply to apply radio frequency current to the cannula. First and second RF electrode plates are provided at an outer portion of an inner insulator formed on an outer circumference of a cannula body except for a cannula tip formed at an end portion of the cannula in opposition to each other. A temperature sensor is interposed between the first and second RF electrode plates, and an outer insulator is formed on an outer peripheral portion of the first and second RF electrode plates and the temperature sensor.

In addition, first and second electrodes are wound around an outer circumference of an inner insulator of the cannula while being spaced apart from each other by a predetermined interval.

As described above, according to the present invention, the cannula is inserted into the skin of the human body. The RF current is applied through the first and second electrodes installed in a cannula body, so that deep heat is generated at a bipolar mode and the fat melted by the deep heat is sucked/discharged out. Therefore, as RF current is applied through a plurality of bipolar electrodes, the RF current can be applied into the biological tissue. In addition, an interval between the first and second RF electrodes, at which the RF current is applied, is short, so that a greater amount of current can be applied to the biological tissue within a short time. Therefore, a greater amount of current can be concentratedly applied within a short time.

Therefore, the melting and sucking of the fatty tissue can simultaneously performed a wide area within a short time, so that the removal of the fatty tissue can be more rapidly and efficiently accelerated.

### BRIEF DESCRIPTION OF THE DRAWINGS

FIG. 1 is an exploded perspective view showing a cannula according to the present invention.

FIG. 2 is an assembling perspective view showing the cannula according to the present invention.

FIG. 3 is an enlarged perspective view showing an electrode plate and an external insulator of FIG. 1.

FIG. 4 is a longitudinal sectional view showing the cannula according to the present invention.

FIG. 5 is a schematic view showing a cannula according to the embodiment used without the external insulator.

FIG. 6 is a schematic view showing the cannula according to the embodiment used without the external insulator.

FIG. 7 is a perspective view showing a cannula according to another embodiment of the present invention.

### DETAILED DESCRIPTION OF THE INVENTION

Hereinafter, embodiments of the present invention will be described in detail with reference to accompanying drawings.

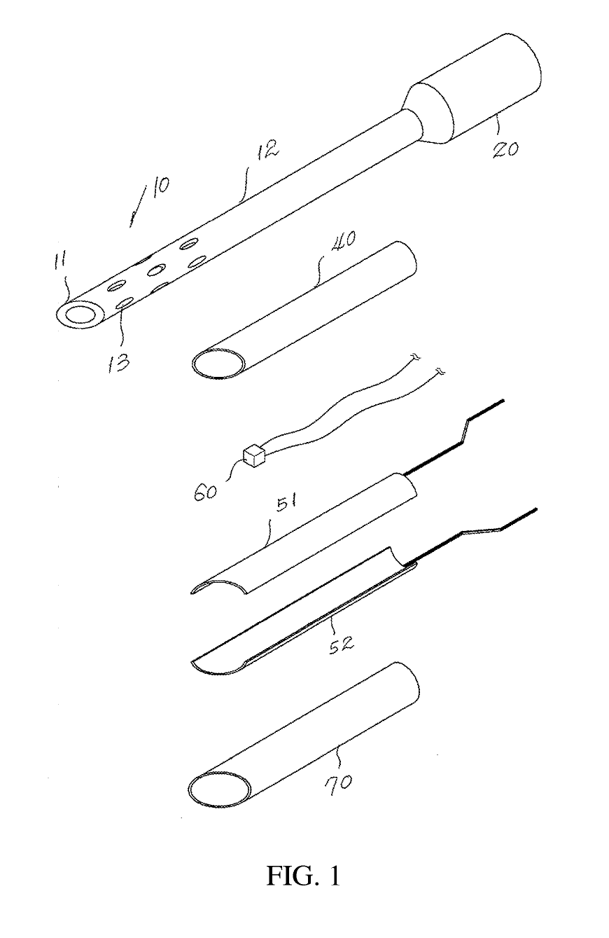

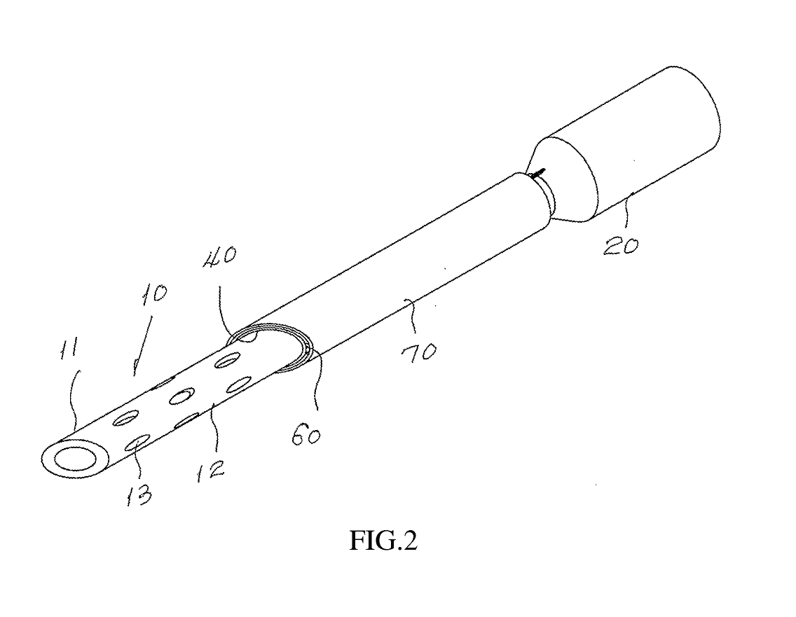

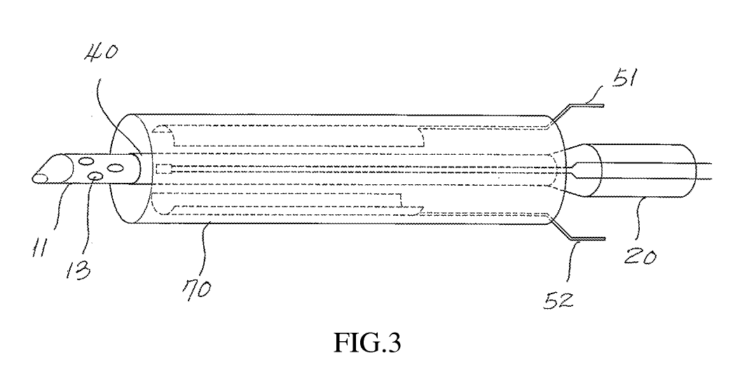

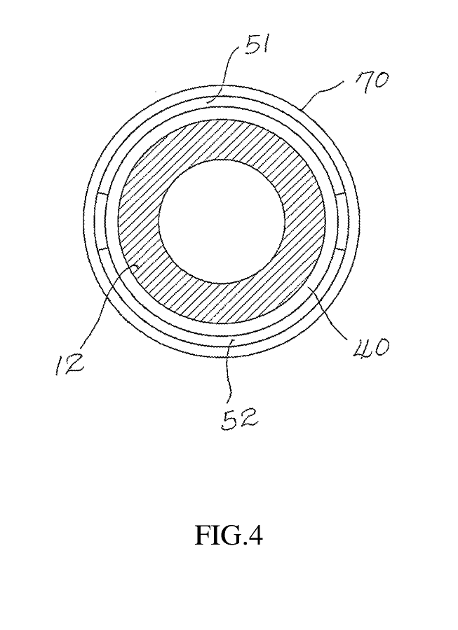

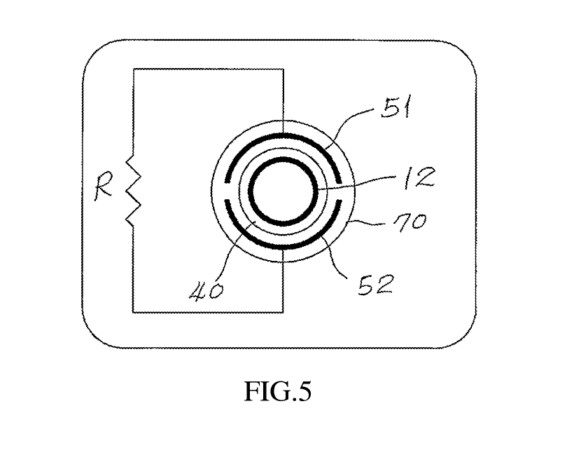

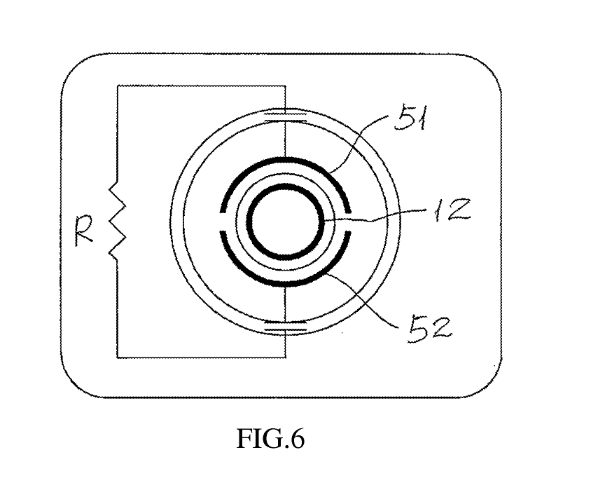

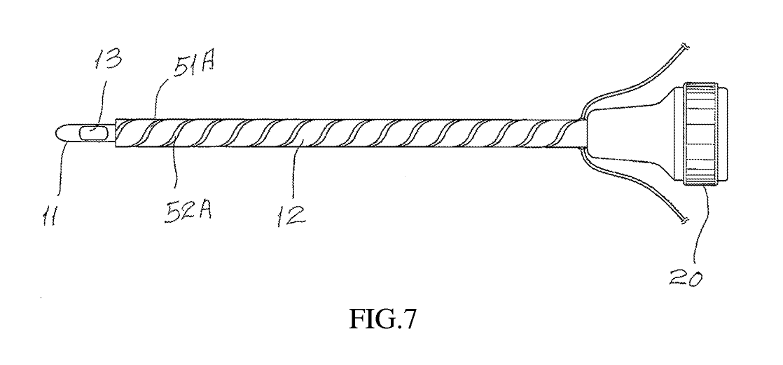

FIG. 1 is an exploded perspective view showing a cannula according to the present invention. FIG. 2 is an assembling perspective view showing the cannula according to the present invention. FIG. 3 is an enlarged perspective view showing an electrode plate and an external insulator of FIG. 1. FIG. 4 is a longitudinal sectional view showing the cannula according to the present invention. FIG. 5 is a schematic view showing the cannula according to the embodiment used without the external insulator. FIG. 6 is a schematic view showing the cannula according to the embodiment used without the external insulator. FIG. 7 is a perspective view showing a cannula according to another embodiment of the present invention.

Hereinafter, a cannula 10 according to a first embodiment of the present invention will be described.

The cannula 10 has an empty space therein and a pipe shape in order to suck a fat in a skin. The cannula 10 has a rear end coupled with a handpiece 20. A control box (not shown) equipped with a micro-controller and a power supply is provided to apply radio frequency (RF) current to the cannula 10. First and second RF electrode plates 51 and 52 are provided at an outer portion of an inner insulator 40 formed on an outer circumference of a cannula body 12 except for a cannula tip 11 formed at an end portion of the cannula 10 in opposition to each other. A temperature sensor 60 is interposed between the first and second RF electrode plates 51 and 52, and an outer insulator 70 is formed on an outer peripheral portion of the first and second RF electrode plates 51 and 52 and the temperature sensor 60.

The first and second RF electrode plates 51 and 52 preferably include an alloy or a nickel plated plate having the same level as that of medical equipment. The first and second RF electrode plates 51 and 52 may be bonded to the inner insulator 40 using an adhesive, or be used through an etching scheme or a deposition scheme.

The cannula tip 11 is formed therein with at least one fat suction hole 13 generally known to those skilled in the art. A suction unit (not shown) including a suction tube and a suction pump is installed at an end portion or an intermediate portion thereof to suck/discharge dissolved fat.

According to another embodiment of the present invention, as shown in FIG. 7, if higher RF energy is required, the interval between electrodes may be reduced and first and second electrodes 51A and 52A are alternately wound in a spiral shape around the outer circumference of the inner insulator 40 of the cannula while being spaced apart from each other by a predetermined interval.

The first and second electrodes 51A and 52A preferably include an alloy or a nickel plated plate having the same level as that of medical equipment. The first and second RF electrode plates 51A and 52A may be bonded to the inner insulator 40 using an adhesive, or be used through an etching scheme or a deposition scheme.

According to the present invention having the above structure, the first and second electrode plates 51 and 52 are installed closely to each other in opposition to each other, so that the first and second electrodes plates 51 and 52 are operated at a bipolar type, that is, a bipolar mode to apply RF current. In this case, RF current having a polarity changed according to the frequency of AC current is applied to the first and second RF electrodes 51 and 52. When comparing with a monopolar type of electrodes, current can be more concentrated between the first and second RF electrodes 51 and 52.

In other words, since an interval between the first and second RF electrodes 51 and 52, at which RF current is applied, is short, a greater amount of current can be applied to a biological tissue, so that a greater amount of current can be applied within a short time.

Therefore, a procedure is performed with respect to a wider biological tissue within the short time, and the melting and sucking of a fatty tissue can simultaneously performed, so that the removal of the fatty tissue can be more rapidly and efficiently accelerated.

Meanwhile, according to the present invention, when the cannula 10 is used without the external insulator 70, since the first and second RF electrode plates 51 and 52 include the alloy having the same level as that of medical equipment, a malicious influence is not exerted on a human organism (marked by resistor R). Accordingly, RF energy can be directly transmitted into the human organism. In addition, when the external insulator 70 is used in contact with the inner part of the human body, both of the first and second RF electrodes 51 and 52 can be used through a capacitive energy transmission scheme that does not require the direct contact with the human organism.

In addition, as described above, a pair of first and second electrodes 51A and 52A are alternately wound in a spiral shape around the outer circumference of the inner insulator 40 of the cannula 10 while being spaced apart from each other by a predetermined distance, so that higher RF energy can be supplied. In addition, since the cannula 10 inserted into the human organism has a small diameter, a plurality of electrodes can be easily installed in the cannula 10 having a small diameter.

Similarly to the first embodiment, the present embodiment may employ a scheme in which the RF electrodes directly make contact with the human organism or does not directly make contact with the human organism.

Meanwhile, according to the present invention, the deep heat in the human organism is sensed by the temperature sensor 60, so that proper RF energy can be applied to the human organism through a program of the micro-controller provided in the control box.

Although a preferred embodiment of the present invention has been described for illustrative purposes, those skilled in the art will appreciate that various modifications, additions and substitutions are possible, without departing from the scope and spirit of the invention as disclosed in the accompanying claims.

What is claimed is:

1. A cannula (10) having an empty space therein and a pipe shape in order to suck a fat in a skin of a human body, the cannula (10) comprising:
a handpiece (20) coupled with a rear end coupled with the cannula (10); and
a control box equipped with a micro-controller and a power supply to apply radio frequency current to the cannula (10),
wherein a pair of first and second electrodes (51A and 52A) are alternately wound in a spiral shape around an outer circumference of an inner insulator (40) of the cannula (10) while being spaced apart from each other by a predetermined interval.
